(19) United States
(12) Patent Application Publication
(10) Pub. No.: US 2021/0285954 A1
(43) Pub. Date: Sep. 16, 2021

(54) ASTROCYTE-DERIVED EXOSOME COMPLEMENT PROTEIN ASSAY FOR TRAUMATIC BRAIN INJURY AND METHODS AND AGENTS FOR TREATING TRAUMATIC BRAIN INJURY

(71) Applicant: Edward J. Goetzl, San Francisco, CA (US)

(72) Inventor: Edward J. Goetzl, San Francisco, CA (US)

(21) Appl. No.: 17/194,285

(22) Filed: Mar. 7, 2021

Related U.S. Application Data
(60) Provisional application No. 62/986,658, filed on Mar. 7, 2020.

(51) Int. Cl. G01N 33/68; G01N 33/563

(52) U.S. Cl. CPC G01N 33/68; G01N 33/563

(57) ABSTRACT

The present invention relates to plasma astrocyte-derived exosomal complement protein biomarkers and diagnostic and prognostic methods for traumatic brain injury (TBI). The invention also provides compositions for detecting plasma astrocyte-derived exosomal complement protein biomarkers as well as compositions and methods useful for treating traumatic brain injury.

### RELATED APPLICATIONS

This application claims priority to the U.S. Provisional Patent Application Ser. No. 62/986,658, filed on Mar. 7, 2020, which is hereby incorporated by reference herein in its entirety.

### FIELD OF THE INVENTION

The present invention relates to plasma astrocyte-derived exosomal complement protein biomarkers and diagnostic and prognostic methods for traumatic brain injury (TBI). The invention also provides compositions for detecting plasma astrocyte-derived exosomal complement protein biomarkers as well as compositions and methods useful for treating traumatic brain injury.

### BACKGROUND OF THE INVENTION

Astrocytes are abundant glial cells in the human central nervous system (CNS) that normally support neurons through promotion of development, nutrition, survival, dendrite outgrowth, and synapse formation (Sofroniew et al. (2010) Acta Neuropathol 119, 7-35; Zamanian et al. (2012) J Neurosci 32, 6391-6410; and Anderson et al. (2016) Nature 532, 195-200). CNS responses to neuroinflammatory and neurodegenerative diseases include elevation in the total number of astrocytes and their increased differentiation into A1-type reactive astrocytes (Ben Haim et al. (2015) Front Cell Neurosci 9, 278; Goetzl et al. (2017) Faseb J 31, 1792-1795; and Liddelow et al. (2017) Immunity 46, 957-967). An ensemble of immune cytokines are prominent inducers of A1-type astrocytes (Choi et al. (2014) PLoS One 9, e92325; Crotti et al. (2016) Immunity 44, 505-515; and Liddelow et al. (2017) Nature 541, 481-487). A1-type astrocytes exhibit increased expression of proinflammatory pathways as well as neuron-directed toxic activities that damage synapses early and later destroy neurons (Zamanian et al. (2012) J Neurosci 32, 6391-6410; Liddelow et al. (2017) Immunity 46, 957-967; Sofroniew (2014) Neuroscientist 20, 160-172; and Ceyzeriat et al. (2016) Neuroscience 330, 205-218).

Studies of postmortem brain tissues from patients with neurodegenerative diseases have delineated some putatively neuron-toxic factors in A1-type astrocytes, but it is not clear which are involved in pathogenesis. Approximately 60% of glial fibrillary acidic protein-positive A1-type astrocytes in the prefrontal cortex of patients with Alzheimer disease (AD) contain a prominently elevated level of complement component 3 (C3) and C3 fragments, which have potential neuronally toxic activity (Ben Haim et al. (2015) Front Cell Neurosci 9, 278 and Liddelow et al. (2017) Immunity 46, 957-967). Evidence of the possible pathogenic involvement of complement systems in AD had been presented, but it was not clear that astrocytes are the principal source of these complement mediators (Ben Haim et al. (2015) Front Cell Neurosci 9, 278; Stevens et al. (2007) Cell 131, 1164-1178; Lian et al. (2015) Neuron 85, 101-115; and Hong et al. (2016) Science 352, 712-716). Finding that reactive astrocytes and complement may protect neurons from proteinopathic factors in some animal models of AD further complicated interpretation of the role of complement in pathogenesis (Kraft et al. (2013) Faseb J 27, 187-198 and Heppner et al. (2015) Nat Rev Neurosci 16, 358-372).

Characterization of astrocyte-derived exosomes (ADEs) enriched from human plasma by sequential precipitation and immunochemical absorption showed much higher levels of the astrocyte biomarkers glutamine synthetase and glial fibrillary acidic protein than in neuron-derived exosomes (NDEs) enriched from the same plasmas (Goetzl et al. (2016) Faseb J 30, 3853-3859). In contrast, NDEs had much higher levels than ADEs of the neuronal markers neurofilament light chain and neuron-specific enolase (Goetzl et al. (2016) Faseb J 30, 3853-3859). Initial analyses of ADEs showed higher levels of β-site amyloid precursor protein-cleaving enzyme 1 (BACE 1) and soluble amyloid precursor protein β of the Aβ42 peptide-generating system than in NDEs (Goetzl et al. (2016) Faseb J 30, 3853-3859).

Quantification of plasma ADE levels of complement proteins provided evidence for increased activation of the classical pathway and alternative amplification loop, but not the lectin pathway, in astrocytes of patients with AD compared to matched controls (Goetzl et al. (2018) Ann Neurol 83, 544-552). Further, ADE content of the complement-regulatory membrane proteins CD59, CD46, decay-accelerating factor, and complement receptor type 1 (CR1), but not of fluid-phase factor I, were significantly lower in AD than controls (Goetzl et al. (2018) Ann Neurol 83, 544-552). These data suggested a neuron-toxic opsonic role for C3b and the possibility of direct neuronal membrane attack by C5b-9 terminal complement complex (TCC) in AD. Such complement abnormalities were detected in the phase of mild cognitive impairment several years preceding dementia and in clinically-evident mild AD, but not in preclinical AD 5-12 years before memory loss (Goetzl et al. (2018) Ann Neurol 83, 544-552 and Winston et al. (2019) Alzheimers Dement (Amst) 11, 61-66). Less is known of the activation and pathogenic involvement of complement systems in TBI than in degenerative neurological diseases.

Thus, there is a need in the art for biomarkers and methods for detecting astrocyte-derived exosomal complement biomarkers associated with the pathogenesis of traumatic brain injury (TBI). Additionally, there is a need in the art for compositions for detecting biomarkers as well as compositions and methods useful for treating traumatic brain injury. The present invention meets this need by providing accurate, noninvasive methods for detecting complement biomarkers that are diagnostic for traumatic brain injury. The present invention further provides novel methods, assays, kits, and compositions for diagnosing, prognosing, predicting, and treating traumatic brain injury.

This background information is provided for the purpose of making known information believed by the applicant to be of possible relevance to the present invention. No admission is necessarily intended, nor should be construed, that any of the preceding information constitutes prior art against the present invention.

### SUMMARY OF THE INVENTION

The invention is based on the discovery of biomarkers from astrocyte-derived exosomes that can be used to detect complement system abnormalities associated with pathogenesis of traumatic brain injury. These biomarkers can be used alone or in combination with one or more additional biomarkers or relevant clinical parameters in prognosis, diagnosis, or monitoring treatment of complement system abnormalities associated with traumatic brain injury. The invention is also based on the discovery that complement inhibitors are useful for treating traumatic brain injury.

Biomarkers that can be used in the practice of the invention include, but are not limited to, complement proteins including, such as, for example, effector proteins (e.g., C3b), membrane-associated complement regulatory proteins (CD59), alternative pathway proteins (e.g., factor D), classical pathway proteins (e.g., C4b), and lectin pathway proteins (e.g., MBL). In some embodiments, biomarkers include, human tetraspanning exosome marker CD81, complement fragment C4b, glutamine synthetase, complement receptor type 1 (CR1), factor I, glial acidic fibrillary protein, complement fragment C3b, complement factor B. Bb fragment of complement factor B, terminal complement complex (TCC) C5b-9, CD59, mannose-binding lectin (MBL), and complement factor D.

In some embodiments, the present invention provides a method comprising: a) providing a biological sample comprising astrocyte-derived exosomes from a subject having or suspected of having a traumatic brain injury; b) enriching the sample for astrocyte-derived exosomes; and c) detecting the presence of one or more biomarkers selected from the group consisting of an effector complement protein, a membrane-associated complement regulatory protein, an alternative pathway complement protein, a classical pathway complement protein, and/or a lectin pathway protein in the sample, thereby detecting the presence of one or more biomarkers in a biological sample from a subject having or suspected of having a traumatic brain injury. In some embodiments, the one or more marker is human tetraspanning exosome marker CD81, complement fragment C4b, glutamine synthetase, complement receptor type 1 (CR1), factor I, glial acidic fibrillary protein, complement fragment C3b, complement factor B, Bb fragment of complement factor B, terminal complement complex (TCC) C5b-9, CD59, mannose-binding lectin (MBL), and/or complement factor D. In some embodiments, the biological sample is selected from the list consisting of whole blood, plasma, serum, lymph, amniotic fluid, urine, and saliva. In other embodiments, the marker is a full-size marker or a fragment of the full-size marker. In yet other embodiments, the detecting the presence of the marker in the biological sample comprises detecting the amount of the marker in the biological sample. In still other embodiments, the method further comprises the step of determining a treatment course of action based on the detection of the one or more biomarkers. In some embodiments, the traumatic brain injury (TBI) is acute TBI, chronic TBI, military TBI, and/or sports-related TBI.

In other embodiments, the invention provides a method comprising: a) providing a biological sample comprising astrocyte-derived exosomes from a subject having a traumatic brain injury or suspected of having a traumatic brain injury; b) isolating astrocyte-derived exosomes from the biological sample; and c) detecting the presence of one or more biomarkers selected from the group consisting of an effector complement protein, a membrane-associated complement regulatory protein, an alternative pathway complement protein, a classical pathway complement protein, and/or a lectin pathway protein in the exosomes. In other embodiments, the isolating astrocyte-derived exosomes from the biological sample comprises: contacting the biological sample with an agent under conditions wherein an astrocyte-derived exosome present in the biological sample binds to the agent to form an astrocyte-derived exosome-agent complex; and isolating the astrocyte-derived exosome from the astrocyte-derived exosome-agent complex to obtain a sample containing the astrocyte-derived exosome, wherein the purity of the astrocyte-derived exosomes present in said sample is greater than the purity of the astrocyte-derived exosomes present in said biological sample. In yet other embodiments, the one or more marker is human tetraspanning exosome marker CD81, complement fragment C4b, glutamine synthetase, complement receptor type 1 (CR1), factor I, glial acidic fibrillary protein, complement fragment C3b, complement factor B. Bb fragment of complement factor B, terminal complement complex (TCC) C5b-9, CD59, mannose-binding lectin (MBL), and/or complement factor D. In still other embodiments, the agent is an antibody. In some embodiments, the antibody is an anti-Glutamine Aspartate Transporter antibody. In other embodiments, the biological sample is selected from the list consisting of whole blood, plasma, serum, lymph, amniotic fluid, urine, and saliva. In yet other embodiments, the marker is a full-size marker or a fragment of the full-size marker. In still other embodiments, the detecting the presence of the marker in the biological sample comprises detecting the amount of the marker in the biological sample. In other embodiments, the methods further comprise the step of determining a treatment course of action based on the detection of the one or more biomarkers. In still other embodiments, the traumatic brain injury (TBI) is acute TBI, chronic TBI, military TBI, and/or sports-related TBI.

In other embodiments, the invention provides methods for treating a subject, comprising the steps of: providing a biological sample from a subject having or suspected of having a traumatic brain injury, wherein the sample comprises astrocyte-derived exosomes; measuring the level of one or more biomarkers selected from the group consisting of an effector complement protein, a membrane-associated complement regulatory protein, an alternative pathway complement protein, a classical pathway complement protein, and/or a lectin pathway protein from the biological sample, wherein an altered level of the one or more biomarkers in the sample relative to the level in a control sample is indicative of a need for treatment; and administering an effective amount of an agent to the subject thereby treating the traumatic brain injury in the subject. In some embodiments, the one or more marker is human tetraspanning exosome marker CD81, complement fragment C4b, glutamine synthetase, complement receptor type 1 (CR1), factor I, glial acidic fibrillary protein, complement fragment C3b, complement factor B, Bb fragment of complement factor B, terminal complement complex (TCC) C5b-9, CD59, mannose-binding lectin (MBL), and/or complement factor D. In other embodiments, the traumatic brain injury (TBI) is acute TBI, chronic TBI, military TBI, and/or sports-related TBI. In yet other embodiments, the agent is a complement pathway inhibitor. In some embodiments, the complement pathway inhibitor is a neutralizing monoclonal antibody to effector complement components or their receptors, decoy complement receptors or receptor antagonists, or an esterase inhibitor.

In other embodiments, the present invention provides a method of detecting markers in a biological sample, the method comprising: a) providing; i) a biological sample comprising astrocyte-derived exosomes from a subject and ii) immunoassay reagents for detection of one or more biomarkers selected from the group consisting of an effector complement protein, a membrane-associated complement regulatory protein, an alternative pathway complement protein, a classical pathway complement protein, and/or a lectin pathway protein; b) isolating astrocyte-derived exosomes from the biological sample and c) detecting the presence of one or more biomarkers selected from the group consisting of CD81, complement fragment C4b, glutamine synthetase, complement receptor type 1 (CR1), factor 1, glial acidic fibrillary protein, complement fragment C3b, complement factor B, Bb fragment of complement factor B, terminal complement complex (TCC) C5b-9, CD59, mannose-binding lectin (MBL), and/or complement factor D in the exosomes using said reagents. In some embodiments, the subject has a traumatic brain injury.

In some embodiments, the reagents comprise antibodies for performing an immunoassay. In some embodiments, the immunoassay is selected from the group consisting of an ELISA, radio-immunoassay, automated immunoassay, cytometric bead assay, and immunoprecipitation assay. In other embodiments, the biological sample can be any bodily fluid comprising astrocyte-derived exosomes, including, but not limited to, whole blood, plasma, serum, lymph, amniotic fluid, urine, and saliva. In some embodiments, the marker is a full-size marker. In other embodiments said marker is a fragment of the full-size marker. In other embodiments, the detecting the presence of the marker in the biological sample comprises detecting the amount of the marker in the biological sample. In some embodiments, the method further comprises the step of determining a treatment course of action based on the detection of the marker or the diagnosis of a traumatic brain injury.

In some embodiments, the subject has been diagnosed with traumatic brain injury or suspected of having a traumatic brain injury. In other embodiments, the subject is at-risk of developing a traumatic brain injury (TBI). In other embodiments, the traumatic brain injury (TBI) is acute TBI, chronic TBI, military TBI, and/or sports-related TBI.

In some embodiments, isolating astrocyte-derived exosomes from the biological sample comprises: contacting the biological sample with an agent under conditions wherein an astrocyte-derived exosome present in the biological sample binds to the agent to form an astrocyte-derived exosome-agent complex; and isolating the astrocyte-derived exosome from the astrocyte-derived exosome-agent complex to obtain a sample containing the astrocyte-derived exosome, wherein the purity of the astrocyte-derived exosomes present in said sample is greater than the purity of the astrocyte-derived exosomes present in said biological sample. The agent may be an antibody that specifically binds to an astrocyte-derived exosome surface marker (e.g., Glutamine Aspartate Transporter (GLAST)). In some aspects of the present embodiment, the contacting comprises incubating or reacting. Example 1 describes isolation of astrocyte-derived exosomes from a biological sample, for example, by immunoabsorption using an anti-human Glutamine Aspartate Transporter (GLAST) (ACSA-1) biotinylated antibody specific for this surface protein.

Biomarker proteins can be measured, for example, by performing immunohistochemistry, immunocytochemistry, immunofluorescence, immunoprecipitation, Western blotting, or an enzyme-linked immunosorbent assay (ELISA). In certain embodiments, the level of a biomarker is measured with an immunoassay. For example, the level of the biomarker can be measured by contacting an antibody with the biomarker, wherein the antibody specifically binds to the biomarker, or a fragment thereof containing an antigenic determinant of the biomarker. Antibodies that can be used in the practice of the invention include, but are not limited to, monoclonal antibodies, polyclonal antibodies, chimeric antibodies, recombinant fragments of antibodies, Fab fragments, Fab′ fragments, F(ab′)₂ fragments, Fᵥ fragments, or scFᵥ fragments. In one embodiment, the method comprises measuring amounts of an in vitro complex comprising a labeled antibody bound to an astrocyte-derived exosome biomarker. In one aspect, the astrocyte-derived exosome biomarker is selected from the group consisting of an effector complement protein, a membrane-associated complement regulatory protein, an alternative pathway complement protein, a classical pathway complement protein, and/or a lectin pathway protein. In other embodiments, the biomarker is human tetraspanning exosome marker CD81, complement fragment C4b, glutamine synthetase, complement receptor type 1 (CR1), factor I, glial acidic fibrillary protein, complement fragment C3b, complement factor B, Bb fragment of complement factor B, terminal complement complex (TCC) C5b-9, CD59, mannose-binding lectin (MBL), and complement factor D. In some embodiments, increased levels of the biomarker human tetraspanning exosome marker CD81, complement fragment C4b, glutamine synthetase, complement receptor type 1 (CR1), factor I, glial acidic fibrillary protein, complement fragment C3b, complement factor B, Bb fragment of complement factor B, terminal complement complex (TCC) C5b-9, CD59, mannose-binding lectin (MBL), and complement factor D compared to reference value ranges of the biomarkers for a control subject indicate that the subject has a traumatic brain injury or is at-risk of developing a traumatic brain injury. In some aspects, the control subject is a subject without a traumatic brain injury. In some embodiments, decreased levels of the biomarker human tetraspanning exosome marker CD81, complement fragment C4b, glutamine synthetase, complement receptor type 1 (CR1), factor I, glial acidic fibrillary protein, complement fragment C3b, complement factor B, Bb fragment of complement factor B, terminal complement complex (TCC) C5b-9, CD59, mannose-binding lectin (MBL), and complement factor D compared to reference value ranges of the biomarkers for a control subject indicate that the subject has a traumatic brain injury or is at-risk of developing a traumatic brain injury. In some aspects, the control subject is a subject without a traumatic brain injury.

The levels of the biomarkers from astrocyte-derived exosomes from a subject can be compared to reference value ranges for the biomarkers found in one or more samples of astrocyte-derived exosomes from one or more subjects without a traumatic brain injury (e.g., control sample, healthy subject without TBI). Alternatively, the levels of the biomarkers from astrocyte-derived exosomes from a subject can be compared to reference values ranges for the biomarkers found in one or more samples of astrocyte-derived exosomes from one or more subjects with a traumatic brain injury. In some embodiments, the traumatic brain injury (TBI) is acute TBI, chronic TBI, military TBI, and/or sports-related TBI.

In some embodiments, the invention provides a method for monitoring the efficacy of a therapy for treating a traumatic brain injury in a patient, the method comprising: a) providing a first biological sample comprising astrocyte-derived exosomes from the patient before the patient undergoes the therapy and a second biological sample comprising astrocyte-derived exosomes after the patient undergoes the therapy; b) isolating astrocyte-derived exosomes from the first biological sample and the second biological sample; and c) detecting one or more biomarkers selected from the group consisting of an effector complement protein, a membrane-associated complement regulatory protein, an alternative pathway complement protein, a classical pathway complement protein, and/or a lectin pathway protein in the astrocyte-derived exosomes from the first biological sample and the second biological sample; and d) comparing the levels of the one or more biomarkers for the astrocyte-derived exosomes from the first biological sample to the levels of the one or more biomarkers for the astrocyte-derived exosomes from the second biological sample, wherein decreased levels of the one or more biomarkers for the astrocyte-derived exosomes from the second biological sample compared to the levels of the one or more biomarkers for the astrocyte-derived exosomes from the first biological sample indicate that the patient is improving, and increased levels of the one or more biomarkers for the astrocyte-derived exosomes from the second biological sample compared to the levels of the one or more biomarkers for the astrocyte-derived exosomes from the first biological sample indicate that the patient is worsening or not responding to the therapy. In some embodiments, the one or more biomarker is human tetraspanning exosome marker CD81, complement fragment C4b, glutamine synthetase, complement receptor type 1 (CR1), factor I, glial acidic fibrillary protein, complement fragment C3b, complement factor B, Bb fragment of complement factor B, terminal complement complex (TCC) C5b-9, CD59, mannose-binding lectin (MBL), and complement factor D.

In some embodiments, the invention provides a method for monitoring the efficacy of a therapy for treating a traumatic brain injury in a patient, the method comprising: a) providing a first biological sample comprising astrocyte-derived exosomes from the patient before the patient undergoes the therapy and a second biological sample comprising astrocyte-derived exosomes after the patient undergoes the therapy; b) isolating astrocyte-derived exosomes from the first biological sample and the second biological sample; and c) detecting one or more biomarkers selected from the group consisting of an effector complement protein, a membrane-associated complement regulatory protein, an alternative pathway complement protein, a classical pathway complement protein, and/or a lectin pathway protein from the astrocyte-derived exosomes from the first biological sample and the second biological sample; and d) comparing the levels of the one or more biomarkers for the astrocyte-derived exosomes from the first biological sample to the levels of the one or more biomarkers for the astrocyte-derived exosomes from the second biological sample, wherein increased levels of the one or more biomarkers for the astrocyte-derived exosomes from the second biological sample compared to the levels of the one or more biomarkers for the astrocyte-derived exosomes from the first biological sample indicate that the patient is improving, and decreased levels of the one or more biomarkers for the astrocyte-derived exosomes from the second biological sample compared to the levels of the one or more biomarkers for the astrocyte-derived exosomes from the first biological sample indicate that the patient is worsening or not responding to the therapy. In some embodiments, the one or more biomarkers are human tetraspanning exosome marker CD81, complement fragment C4b, glutamine synthetase, complement receptor type 1 (CR1), factor I, glial acidic fibrillary protein, complement fragment C3b, complement factor B, Bb fragment of complement factor B, terminal complement complex (TCC) C5b-9, CD59, mannose-binding lectin (MBL), and/or complement factor D.

In other embodiments, the invention provides a method for monitoring traumatic brain injury in a subject, the method comprising: a) measuring levels of one or more biomarkers selected from the group consisting of an effector complement protein, a membrane-associated complement regulatory protein, an alternative pathway complement protein, a classical pathway complement protein, and/or a lectin pathway protein from astrocyte-derived exosomes from a first biological sample from the subject, wherein the first biological sample is obtained from the subject at a first time point; b) measuring levels of one or more biomarkers selected from the group consisting of an effector complement protein, a membrane-associated complement regulatory protein, an alternative pathway complement protein, a classical pathway complement protein, and/or a lectin pathway protein from astrocyte-derived exosomes from a second biological sample from the subject, wherein the second biological sample is obtained from the subject at a second (i.e., later) time point; and c) comparing the levels of the biomarkers for astrocyte-derived exosomes from the first biological sample to the levels of the biomarkers for astrocyte-derived exosomes from the second biological sample, wherein decreased levels of the one or more biomarkers selected from the group consisting of an effector complement protein, a membrane-associated complement regulatory protein, an alternative pathway complement protein, a classical pathway complement protein, and/or a lectin pathway protein from the astrocyte-derived exosomes from the second biological sample compared to the levels of the biomarkers in the first biological sample indicate that the patient is improving, and increased levels of the one or more biomarkers selected from the group consisting of an effector complement protein, a membrane-associated complement regulatory protein, an alternative pathway complement protein, a classical pathway complement protein, and/or a lectin pathway protein from the astrocyte-derived exosomes from the second biological sample compared to the levels of the biomarkers for the astrocyte-derived exosomes from the first biological sample indicate that the patient is worsening. In some embodiments, the one or more biomarkers are selected from the group consisting of human tetraspanning exosome marker CD81, complement fragment C4b, glutamine synthetase, complement receptor type 1 (CR1), factor I, glial acidic fibrillary protein, complement fragment C3b, complement factor B, Bb fragment of complement factor B, terminal complement complex (TCC) C5b-9, CD59, mannose-binding lectin (MBL), and complement factor D.

In other embodiments, the invention provides a method for monitoring traumatic brain injury in a subject, the method comprising: a) measuring levels of one or more biomarkers selected from the group consisting of an effector complement protein, a membrane-associated complement regulatory protein, an alternative pathway complement protein, a classical pathway complement protein, and a lectin pathway protein from astrocyte-derived exosomes from a first biological sample from the subject, wherein the first biological sample is obtained from the subject at a first time point; b) measuring levels of one or more biomarkers selected from the group consisting of an effector complement protein, a membrane-associated complement regulatory protein, an alternative pathway complement protein, a classical pathway complement protein, and a lectin pathway protein from astrocyte-derived exosomes from a second biological sample from the subject, wherein the second biological sample is obtained from the subject at a second (i.e., later) time point; and c) comparing the levels of the biomarkers for astrocyte-derived exosomes from the first biological sample to the levels of the biomarkers for astrocyte-derived exosomes from the second biological sample, wherein increased levels of the one or more biomarkers selected from the group consisting of an effector complement protein, an alternative pathway complement protein, a classical pathway complement protein, and a lectin pathway protein from the astrocyte-derived exosomes from the second biological sample compared to the levels of the biomarkers in the first biological sample indicate that the patient is improving, and decreased levels of the one or more biomarkers comprising a membrane-associated complement regulatory protein from the astrocyte-derived exosomes from the second biological sample compared to the levels of the biomarkers for the astrocyte-derived exosomes from the first biological sample indicate that the patient is worsening.

In yet other embodiments, the invention provides a method of treating a patient suspected of having a traumatic brain injury, the method comprising: a) detecting astrocyte-exosomal biomarker levels in the patient or receiving information regarding the astrocyte-exosomal biomarker levels of the patient, as determined according to a method described herein; and b) administering a therapeutically effective amount of at least one agent that alters astrocyte-exosomal biomarker levels in the subject. After treatment, the method may further comprise monitoring the response of the patient to treatment. In some embodiments, the agent is a complement pathway inhibitor. In other embodiments, the complement pathway inhibitor is a neutralizing monoclonal antibody to effector complement components or their receptors, decoy complement receptors or receptor antagonists, or an esterase inhibitor.

In other embodiments, the invention provides a method comprising: providing a biological sample from a subject suspected of having a traumatic brain injury; detecting the presence or level of at least one or more biomarkers selected from the group consisting of an effector complement protein, a membrane-associated complement regulatory protein, an alternative pathway complement protein, a classical pathway complement protein, and a lectin pathway protein; and administering a treatment to the subject. In one embodiment, the method further comprises administering a therapeutically effective amount of at least one agent that treats traumatic brain injury to the subject if increased levels of the one or more biomarkers are detected in the subject. In one embodiment, the method further comprises administering a therapeutically effective amount of at least one agent that treats traumatic brain injury to the subject if decreased levels of the one or more biomarkers are detected in the subject. After treatment, the method may further comprise monitoring the response of the subject to treatment. In some embodiments, the one or more biomarkers comprises human tetraspanning exosome marker CD81, complement fragment C4b, glutamine synthetase, complement receptor type 1 (CR1), factor I, glial acidic fibrillary protein, complement fragment C3b, complement factor B. Bb fragment of complement factor B, terminal complement complex (TCC) C5b-9, CD59, mannose-binding lectin (MBL), and/or complement factor D.

In other embodiments, the present invention provides a method of treating a subject with traumatic brain injury, comprising: providing a biological sample from the subject; determining the level of at least one or more biomarkers selected from the list consisting of an effector complement protein, a membrane-associated complement regulatory protein, an alternative pathway complement protein, a classical pathway complement protein, and a lectin pathway protein using at least one reagent that specifically binds to said biomarkers; and prescribing a treatment regimen based on the level of the one or more biomarkers. In some embodiments, the method further comprises isolating astrocyte-derived exosomes from the biological sample. In some embodiments, the biomarker comprises human tetraspanning exosome marker CD81, complement fragment C4b, glutamine synthetase, complement receptor type 1 (CR1), factor I, glial acidic fibrillary protein, complement fragment C3b, complement factor B, Bb fragment of complement factor B, terminal complement complex (TCC) C5b-9. CD59, mannose-binding lectin (MBL), and/or complement factor D

In some embodiments, the invention provides a set of biomarkers for assessing traumatic brain injury status of a subject, the set comprising one or more biomarkers selected from the group consisting of an effector complement protein, a membrane-associated complement regulatory protein, an alternative pathway complement protein, a classical pathway complement protein, and/or a lectin pathway protein, wherein astrocyte-derived exosome levels of the biomarkers in the set are assayed; and wherein the biomarker levels of the set of biomarkers determine the traumatic brain injury status of the subject with at least 40%, 50%, 60%, 70%, 75%, 80%, 85%, 90%, 95%, 99%, or 100% specificity. In some aspects, the set of biomarkers determine the traumatic brain injury status of the subject with at least 40%, 50%, 60%, 70%, 75%, 80%, 85%, 90%, 95%, 99%, or 100% sensitivity. In yet other aspects, the set of biomarkers determine the traumatic brain injury status of the subject with at least 40%, 50%, 60%, 70%, 75%, 80%, 85%, 90%, 95%, 99%, or 100% accuracy. In some embodiments the biomarker comprises human tetraspanning exosome marker CD81, complement fragment C4b, glutamine synthetase, complement receptor type 1 (CR1), factor I, glial acidic fibrillary protein, complement fragment C3b, complement factor B, Bb fragment of complement factor B, terminal complement complex (TCC) C5b-9, CD59, mannose-binding lectin (MBL), and/or complement factor D.

In other embodiments, the invention provides a composition comprising at least one in vitro complex comprising a labeled antibody bound to a biomarker protein selected from the group consisting of an effector complement protein, a membrane-associated complement regulatory protein, an alternative pathway complement protein, a classical pathway complement protein, and a lectin pathway protein, wherein said biomarker protein is extracted from astrocyte-derived exosomes of a subject who has been diagnosed with traumatic brain injury, suspected of having a traumatic brain injury, or at risk of developing a traumatic brain injury. The antibody may be detectably labeled with any type of label, including, but not limited to, a fluorescent label, an enzyme label, a chemiluminescent label, or an isotopic label. In some embodiments, the composition is in a detection device (i.e., device capable of detecting labeled antibody). In some embodiments, the one or more biomarkers comprise human tetraspanning exosome marker CD81, complement fragment C4b, glutamine synthetase, complement receptor type 1 (CR1), factor I, glial acidic fibrillary protein, complement fragment C3b, complement factor B, Bb fragment of complement factor B, terminal complement complex (TCC) C5b-9, CD59, mannose-binding lectin (MBL), and/or complement factor D.

In other embodiments, the invention provides a kit for detecting or monitoring a traumatic brain injury in a subject. In some embodiments, the kit may include a container for holding a biological sample isolated from a subject who has been diagnosed or suspected of having a traumatic brain injury or at risk of developing a traumatic brain injury, at least one agent that specifically detects a biomarker of the present invention; and printed instructions for reacting the agent with astrocyte-derived exosomes from the biological sample or a portion of the biological sample to detect the presence or amount of at least one biomarker. In other embodiments, the kit may also comprise one or more agents that specifically bind astrocyte-derived exosomes for use in isolating astrocyte-derived exosomes from a biological sample. In yet other embodiments, the kit may further comprise one or more control reference samples and reagents for performing an immunoassay. In certain embodiments, the agents may be packaged in separate containers. In some embodiments, the kit comprises agents for measuring the levels of an effector complement protein, a membrane-associated complement regulatory protein, an alternative pathway complement protein, a classical pathway complement protein, and/or a lectin pathway protein. In yet other embodiments, the kit further comprises an antibody that binds to an astrocyte-derived exosome surface marker (e.g., Glutamine Aspartate Transporter (GLAST)).

In other embodiments, the invention provides a method for treating a traumatic brain injury, the method comprising the steps of: providing a biological sample from a subject suspected of having a traumatic brain injury, wherein the sample comprises astrocyte-derived exosomes; measuring the level of one or more biomarkers selected from the group consisting of an effector complement protein, a membrane-associated complement regulatory protein, an alternative pathway complement protein, a classical pathway complement protein, and a lectin pathway protein from the biological sample, wherein an altered level of the one or more biomarkers in the sample relative to the level in a control sample is indicative of a need for treatment; and administering an effective amount of an agent to the subject thereby treating the traumatic brain injury in the subject. In some embodiments, the agent is a recombinant complement control protein selected from the group consisting of recombinant membrane inhibitor of reactive lysis (CD59), recombinant membrane cofactor protein (CD46), recombinant decay-accelerating factor (DAF) and recombinant complement receptor type 1 (CR1). In other embodiments, the agent is neutralizing monoclonal antibodies to effector complement components or their receptors, decoy complement receptors or receptor antagonists, or an esterase inhibitor.

The invention provides methods and compositions for treating traumatic brain injury in a subject. In some embodiments, the traumatic brain injury is sports-related traumatic brain injury (sTBI) or military traumatic brain injury (mTBI). In other embodiments, the traumatic brain injury is acute TBI (i.e., early TBI) or chronic TBI.

The invention provides methods for treating traumatic brain injury in a subject. In some embodiments, the invention provides a method of treating traumatic brain injury in a subject, the method comprising administering an effective amount of a complement pathway inhibitor to a subject having or suspected of having a traumatic brain injury, thereby treating the traumatic brain injury. In other embodiments, the traumatic brain injury (TBI) is acute TBI, chronic TBI, military TBI, and/or sports-related TBI. In yet other embodiments, the complement pathway inhibitor is a neutralizing monoclonal antibody to effector complement components or their receptors, decoy complement receptors or receptor antagonists, or an esterase inhibitor.

These and other embodiments of the present invention will readily occur to those of skill in the art in light of the disclosure herein, and all such embodiments are specifically contemplated.

### INCORPORATION BY REFERENCE

All publications, patents, and patent applications mentioned in this specification are herein incorporated by reference in their entireties to the same extent as if each individual publication, patent, or patent application was specifically and individually indicated to be incorporated by reference.

### BRIEF DESCRIPTION OF THE DRAWINGS

FIGS. 1A-1F set forth data showing astrocyte-derived exosomal (ADE) levels of complement effector proteins in sTBI and mtTBI subject groups. Each point represents the value for a control or TBI participant and the horizontal line in point clusters is the mean level for that group. Mean±S.E.M. for sTBI control, sTBI acute, sTBI chronic, mtTBI control, mtTBI early chronic, and mtTBI late chronic participant values, respectively, are 741±79.6, 501±21.6, 887±74.0, 841±54.9, 483±90.6, and 586±45.9 pg/ml for C4b (A), 1889±65.4, 23,414±2139, 2091±101, 1107±90.6, 15,420±1022, and 3207±289 pg/ml for C3b (B), 45.2±7.25, 629±62.2, 53.4±6.46, 24.3±5.29, 4493±690, and 436±80.5 pg/ml for C5b-9 TCC (C), 741±79.6, 501±21.6, 887±74.0, 841±54.9, 483±90.6, and 586±45.9 pg/ml for factor D (D), 115,423±4298, 491,384±73,188, 206,574±17,089, 103,866±9960, 194,506±27,240, and 140,820120,312 pg/ml for Bb (E), and 1121±82.4, 6899±1112, 2752±363, 829±144, 2109±440, and 955±127 pg/ml for MBL (F). The significance of differences shown between values for controls and TBI subjects were calculated by an unpaired Student's t test; *=p<0.05, *=p<0.01, **=p<0.0001.

FIGS. 2A-2C set forth data showing altered levels of astrocyte-derived exosomal complement protein biomarkers in sTBI and mtTBI patients. Each point represents the value for a control or TBI participant and the horizontal line in point clusters is the mean level for that group. Mean±S.E.M. for sTBI control, sTBI acute, sTBI chronic, mtTBI control, mtTBI early chronic, and mtTBI late chronic participant values, respectively, are 741±79.6, 501±21.6, 887±74.0, 841±54.9, 483±90.6, and 586±45.9 pg/ml for CR1 (A), 1196±157, 740±84.5, 1289±118, 1233±147, 870±76.0, and 1334±97.0 pg/ml for CD59 (B), and 5566±538, 5545±388, 6492±666, 7957±633, 8509±967, and 6640±430 pg/ml for factor I (C). The significance of differences shown between values for controls and TBI subjects were calculated by an unpaired Student's t test; *=p<0.05, *=p<0.01.

Each of the limitations of the invention can encompass various embodiments of the invention. It is, therefore, anticipated that each of the limitations of the invention involving any one element or combinations of elements can be included in each aspect of the invention. This invention is not limited in its application to the details of construction and the arrangement of components set forth in the following description. The invention is capable of other embodiments and of being practiced or of being carried out in various ways. Also, the phraseology and terminology used herein is for the purpose of description and should not be regarded as limiting. The use of “including,” “comprising,” or “having,” “containing”, “involving”, and variations thereof herein, is meant to encompass the items listed thereafter and equivalents thereof as well as additional items. It must be noted that as used herein and in the appended claims, the singular forms “a,” “an,” and “the” include plural references unless context clearly dictates otherwise. Thus, for example, a reference to “a fragment” includes a plurality of such fragments, a reference to an “antibody” is a reference to one or more antibodies and to equivalents thereof known to those skilled in the art, and so forth.

### DESCRIPTION OF THE INVENTION

It is to be understood that the invention is not limited to the particular methodologies, protocols, cell lines, assays, and reagents described herein, as these may vary. It is also to be understood that the terminology used herein is intended to describe particular embodiments of the present invention, and is in no way intended to limit the scope of the present invention as set forth in the appended claims.

Unless defined otherwise, all technical and scientific terms used herein have the same meanings as commonly understood by one of ordinary skill in the art to which this invention belongs. Although any methods and materials similar or equivalent to those described herein can be used in the practice or testing of the present invention, the preferred methods, devices, and materials are now described. All publications cited herein are incorporated herein by reference in their entirety for the purpose of describing and disclosing the methodologies, reagents, and tools reported in the publications that might be used in connection with the invention. Nothing herein is to be construed as an admission that the invention is not entitled to antedate such disclosure by virtue of prior invention.

The practice of the present invention will employ, unless otherwise indicated, conventional methods of chemistry, biochemistry, molecular biology, cell biology, genetics, immunology and pharmacology, within the skill of the art. Such techniques are explained fully in the literature. See, e.g., Gennaro, A. R., ed. (1990) Remington's Pharmaceutical Sciences, 18th ed., Mack Publishing Co.; Colowick, S. et al., eds., Methods In Enzymology, Academic Press, Inc.; Handbook of Experimental Immunology, Vols. I-IV (D. M. Weir and C.C. Blackwell, eds., 1986, Blackwell Scientific Publications); Maniatis, T. et al., eds. (1989) Molecular Cloning: A Laboratory Manual, 2nd edition, Vols. I-III, Cold Spring Harbor Laboratory Press; Ausubel, F. M. et al., eds. (1999) Short Protocols in Molecular Biology, 4th edition, John Wiley & Sons; Ream et al., eds. (1998) Molecular Biology Techniques: An Intensive Laboratory Course, Academic Press); PCR (Introduction to Biotechniques Series), 2nd ed. (Newton & Graham eds., 1997, Springer Verlag).

The present invention relates, in part, to the discovery that astrocyte-derived exosomal biomarkers can be used to detect pathogenesis of traumatic brain injury. The inventor has demonstrated that astrocyte-derived exosome (ADE) levels of complement proteins including, for example, effector proteins (e.g., C3b), membrane-associated complement regulatory proteins (CD59), alternative pathway proteins (e.g., factor D), classical pathway proteins (e.g., C4b), and lectin pathway proteins (e.g., MBL) are altered in subjects with traumatic brain injury. The inventor has identified certain biomarkers, such as, for example, CD81, glutamine synthetase, factor I, glial acidic fibrillary protein, mannose-binding lectin (MBL), inflammatory cytokines, interleukin 6 (IL-6), tumor necrosis factor alpha (TNF-α), interleukin 1 beta (IL-10), complement proteins, C1q, C4b, C5b, C3d, factor B, factor D, Fragment Bb, C3b, C5b-C9 terminal complement complex (TCC), complement regulatory proteins, membrane inhibitor of reactive lysis (CD59), membrane cofactor protein (CD46), decay-accelerating factor (DAF) and complement receptor type 1 (CR1) are altered in subjects with traumatic brain injury (see, e.g., Example 1).

The present invention also provides agents for use in the methods described herein. Such agents may include small molecule compounds; peptides and proteins including antibodies or functionally active fragments thereof.

The present invention further provides kits for identifying a subject at risk of a traumatic brain injury or prescribing a therapeutic regimen or predicting benefit from therapy in a subject having a traumatic brain injury or at risk of developing a traumatic brain injury. In these embodiments, the kits comprise one or more antibodies which specifically bind astrocyte-derived exosomes, one or more antibodies which specifically bind an astrocyte-derived exosomal biomarker of the present invention, one or more containers for collecting and or holding the biological sample, and instructions for the kits use.

The present invention further provides methods for treating traumatic brain injury in a subject. In these embodiments, the invention provides methods for treating traumatic brain injury in a subject comprising administering an effective amount of an agent to the subject, thereby treating the traumatic brain injury in the subject. In some embodiments, the invention provides methods of treating traumatic brain injury in a subject, comprising administering an effective amount of a complement pathway inhibitor to a subject having or suspected of having a traumatic brain injury, thereby treating the traumatic brain injury.

The section headings are used herein for organizational purposes only, and are not to be construed as in any way limiting the subject matter described herein.

### Complement System

The complement system provides an early acting mechanism to initiate and amplify the inflammatory response to microbial infection and other acute insults. While complement activation provides a valuable first-line defense against potential pathogens, the activities of complement that promote a protective inflammatory response can also represent a potential threat to the host. For example, C3 and C5 proteolytic products recruit and activate neutrophils. These activated cells are indiscriminate in their release of destructive enzymes and may cause organ damage. In addition, complement activation may directly cause the deposition of lytic complement components, such as C5b-C9 TCC, on nearby host cells as well as on microbial targets, resulting in host cell lysis. Some products of complement activation, such as C3b, bind to neurons as well as microbes and thereby cause attachment of neuron-destructive CNS cells, such as microglia.

Complement can be activated through either of two distinct enzymatic cascades, referred to as the classical and alternative pathways. The classical pathway is usually triggered by antibody bound to a foreign particle and thus requires prior exposure to that particle for the generation of specific antibody. There are four plasma proteins specifically involved in the classical pathway: C1, C2, C4 and C3. The interaction of C1 with the Fc regions of IgG or IgM in immune complexes activates a C1 protease that can cleave plasma protein C4, resulting in the C4a and C4b fragments. C4b can bind another plasma protein, C2. The resulting species, C4b2, is cleaved by the C1 protease to form the classical pathway C3 convertase, C4b2a. Addition of the C3 cleavage product, C3b, to C3 convertase leads to the formation of the classical pathway C5 convertase, C4b2a3b.

In contrast to the classical pathway, the alternative pathway is spontaneously triggered by foreign or other abnormal surfaces (bacteria, yeast, virally infected cells, or damaged tissue) and is therefore capable of an immediate response to an invading organism. There are four plasma proteins directly involved in the alternative pathway: C3, factors B and D, and properdin (also called factor P). The initial interaction that triggers the alternative pathway is not completely understood. However, it is thought that spontaneously activated C3 (C3b) binds factor B, which is then cleaved by factor D to form the complex C3bBb that possesses C3 convertase activity. The resulting convertase proteolytically modifies additional C3, producing the C3b fragment, which can covalently attach to the target and then interact with factors B and D and form the alternative pathway C3 convertase, C3bBb. The alternative pathway C3 convertase is stabilized by the binding of properdin. However, properdin binding is not required to form a functioning alternative pathway C3 convertase. Since the substrate for the alternative pathway C3 convertase is C3, C3 is therefore both a component and a product of the reaction. As the C3 convertase generates increasing amounts of C3b, an amplification loop is established. In as much as the classical pathway also may generate C3b that can bind factor B, both pathways may amplify activation of the alternative pathway. This allows more C3b to deposit on a target. For example, as described above, the binding of antibody to antigen initiates the classical pathway. If antibodies latch on to bacteria, the classical pathway generates C3b, which couples to target pathogens. However, it has been suggested that from 10% to 90% of the subsequent C3b deposited may come from the alternative pathway. The actual contribution of the alternative pathway to the formation of additional C3b subsequent to classical pathway initiation has not been clearly quantified and thus remains unknown. Addition of C3b to the C3 convertase leads to the formation of the alternative pathway C5 convertase, C3bBbC3b.

Both the classical and alternative pathways involve C3b and converge at C5, which is cleaved to form products with multiple proinflammatory effects. The converged pathway has been referred to as the terminal complement pathway. C5a is the most potent anaphylatoxin, inducing alterations in smooth muscle and vascular tone, as well as vascular permeability. It is also a powerful chemotaxin and activator of both neutrophils and monocytes. C5a-mediated cellular activation can significantly amplify inflammatory responses by inducing the release of multiple additional inflammatory mediators, including cytokines, hydrolytic enzymes, arachidonic acid metabolites and reactive oxygen species. C5 cleavage leads to the formation of C5b-9, also known as the membrane terminal attack complex (MAC or TCC). There is now strong evidence that MAC may play an important role in inflammation in addition to its role as a lytic pore-forming complex. The invention provides methods for detecting astrocyte-derived exosomal levels of complement system proteins. The administration of one or more neutralizing monoclonal antibodies to effector complement components or their receptors, decoy complement receptors or receptor antagonists, and esterase inhibitors of complement mediator generation may suppress ongoing complement-mediated neuronal injury and be useful for treating traumatic brain injury. In some embodiments, the methods of the present invention are used to treat a traumatic brain injury in a subject. In other embodiments, the present invention provides a method for treating a subject having a traumatic brain injury, comprising the steps of: providing a biological sample from a subject having or suspected of having a traumatic brain injury, wherein the sample comprises astrocyte-derived exosomes; measuring the level of one or more biomarkers selected from the group consisting of an effector complement protein, a membrane-associated complement regulatory protein, an alternative pathway complement protein, a classical pathway complement protein, and/or a lectin pathway protein from the biological sample, wherein an altered level of the one or more biomarkers in the sample relative to the level in a control sample is indicative of a need for treatment; and administering an effective amount of an agent to the subject thereby treating the traumatic brain injury in the subject. In certain embodiments, the traumatic brain injury is acute TBI, chronic TBI, military TBI, and/or sports-related TBI.

Many regulatory proteins exist to prevent excessive complement activation and to protect our cells and tissues from damage. The complement system distinguishes self from non-self via a range of specialized cell-surface and soluble proteins. These proteins belong to a family called the regulators of complement activation (RCA) or complement control proteins (CCP). Complement control proteins (or complement regulatory proteins) work in concert to maintain activation of the complement system at a level optimal for host defenses against microbes without damaging host tissues. Many of the complement control proteins act on the convertases, C3b.Bb and C4b.2a, which are bimolecular complexes formed early on in the complement cascade, but CD59 blocks formation of C5b-C9 TCC.

Every cell in the human body is protected by one or more of the membrane-associated RCA proteins, CR1, DAF or MCP. Factor H and C4BP circulate in the plasma and are recruited to self-surfaces through binding to host-specific polysaccharides such as the glycosaminoglycans. Most act to disrupt the formation of the convertases or to shorten the life-span of any complexes that do manage to form. Their presence on self-surfaces, and their absence from the surfaces of foreign particles, means that these regulators perform the important task of targeting complement to where it is needed—on the invading bacterium for example—while preventing activation on host tissues. For example, C3b.Bb is an important convertase that is part of the alternative pathway, and it is formed when factor B binds C3b and is subsequently cleaved. To prevent this from happening, factor H competes with factor B to bind C3b; if it manages to bind, then the convertase is not formed. Factor H can bind C3b much more easily in the presence of sialic acid, which is a component of most cells in the human body; conversely, in the absence of sialic acid, factor B can bind C3b more easily. This means that if C3b is bound to a “self” cell, the presence of sialic acid and the binding of factor H will prevent the complement cascade from activating; if C3b is bound to a bacterium, factor B will bind and the cascade will be set off as normal. The present invention provides methods for detecting astrocyte-derived exosomal levels of complement regulatory proteins. The administration of one or more recombinant complement control proteins early in traumatic brain injury, guided by their levels in astrocyte-derived exosomes of subjects, could limit recruitment of complement mechanisms preventatively. In some embodiments, the methods of the present invention are used to treat a traumatic brain injury in a subject. In other embodiments, the present invention provides a method for treating a subject having a traumatic brain injury, comprising the steps of: providing a biological sample from a subject suspected of having a traumatic brain injury, wherein the sample comprises astrocyte-derived exosomes; measuring the level of one or more biomarkers selected from the group consisting of an effector complement protein, a membrane-associated complement regulatory protein, an alternative pathway complement protein, a classical pathway complement protein, and/or a lectin pathway protein in the sample from the biological sample, wherein an altered level of the one or more biomarkers in the sample relative to the level in a control sample is indicative of a need for treatment; and administering an effective amount of an complement pathway inhibitor to the subject thereby treating the traumatic brain injury in the subject. In certain embodiments, the traumatic brain injury is acute TBI, chronic TBI, military TBI, and/or sports-related TBI. In other embodiments, the complement pathway inhibitor is a neutralizing monoclonal antibody to effector complement components or their receptors, decoy complement receptors or receptor antagonists, or an esterase inhibitor.

### Biological Sample

The present invention provides biomarkers and diagnostic and prognostic methods for traumatic brain injury. Biomarker are detected from astrocyte-derived exosomes from a biological sample obtained from a subject. Biological samples can include any bodily fluid comprising exosomes, including, but not limited to, whole blood, plasma, serum, lymph, amniotic fluid, and saliva.

In some embodiments, the biological sample of the invention can be obtained from blood. In some embodiments, about 1-10 mL of blood is drawn from a subject. In other embodiments, about 10-50 mL of blood is drawn from a subject. Blood can be drawn from any suitable area of the body, including an arm, a leg, or blood accessible through a central venous catheter. In some embodiments, blood is collected following a treatment or activity. For example, blood can be collected following a medical exam. The timing of collection can also be coordinated to increase the number and/or composition of astrocyte-derived exosomes present in the sample. For example, blood can be collected following exercise or a treatment that induces vascular dilation.

Blood may be combined with various components following collection to preserve or prepare samples for subsequent techniques. For example, in some embodiments, blood is treated with an anticoagulant, a cell fixative, a protease inhibitor, a phosphatase inhibitor, or preservative(s) for protein or DNA or RNA following collection. In some embodiments, blood is collected via venipuncture using a needle and a syringe that is emptied into collection tubes containing an anticoagulant such as EDTA, heparin, or acid citrate dextrose (ACD). Blood can also be collected using a heparin-coated syringe and hypodermic needle. Blood can also be combined with components that will be useful for cell culture. For example, in some embodiments, blood is combined with cell culture media or supplemented cell culture media (e.g., cytokines). In certain embodiments, platelet-rich plasma (PRP) is mixed with PBS to block ex vivo platelet activation before centrifugation to yield platelet-poor plasma (PPP).

### Enrichment or Isolation of Astrocyte-Derived Exosomes

Samples can be enriched for astrocyte-derived exosomes through positive selection, negative selection, or a combination of positive and negative selection. In some embodiments, exosomes are directly captured. In other embodiments, blood cells are captured and exosomes are collected from the remaining biological sample.

Samples can also be enriched for exosomes based on the biochemical properties of exosomes. The first step is physical isolation entailing polymer precipitation with centrifugation in one or two cycles. Then, for example, samples can be enriched for exosomes based on differences in antigens. In some of the embodiments, antibody-conjugated magnetic or paramagnetic beads in magnetic field gradients or fluorescently labeled antibodies with flow cytometry are used. In some of the embodiments based on metabolic differences, dye uptake/exclusion measured by flow cytometry or another sorting technology is used. Samples can also be enriched for exosomes based on other biochemical properties known in the art. For example, samples can be enriched for exosomes using ligands or soluble receptors.

In some embodiments, surface markers are used to positively enrich astrocyte-derived exosomes in the sample. In other embodiments, cell surface markers that are not found on exosomes are used to negatively enrich exosomes by depleting cell populations. Modified versions of flow cytometry sorting may also be used to further enrich for astrocyte-derived exosomes using surface markers or intracellular or extracellular markers conjugated to fluorescent labels. Intracellular and extracellular markers may include nuclear stains or antibodies against intracellular or extracellular proteins preferentially expressed in exosomes. Cell surface markers may include cell surface antigens that are preferentially expressed on astrocyte-derived exosomes. In some embodiments, the cell surface marker is an astrocyte-derived exosome surface marker, including, for example, Glutamine Aspartate Transporter (GLAST). In some embodiments, a monoclonal antibody that specifically binds to GLAST (e.g., ACSA-1, mouse anti-human GLAST antibody) is used to enrich or isolate astrocyte-derived exosomes from the sample. In certain aspects, the antibody against GLAST is biotinylated. In this embodiment, the biotinylated antibody can form an antibody-exosome complex that can be subsequently isolated using streptavidin-agarose resin or beads. In other embodiments, the antibody is a monoclonal anti-human GLAST antibody (e.g., ACSA-1).

In other embodiments, astrocyte-derived exosomes are isolated or enriched from a biological sample comprising: contacting a biological sample with an agent under conditions wherein an astrocyte-derived exosome present in said biological sample binds to said agent to form an astrocyte-agent complex; and isolating said exosome from said exosome-agent complex to obtain a sample containing said exosome, wherein the purity of the exosomes present in the sample is greater than the purity of exosomes present in the biological sample. In certain embodiments, the contacting is incubating or reacting. In certain embodiments, the exosomes are astrocyte-derived exosomes. In certain embodiments, the agent is an antibody or a lectin. Lectins useful for forming an exosome-lectin complex are described in U.S. Patent Application Publication No. 2012/0077263. In some embodiments, multiple isolating or enriching steps are performed. In certain aspects of the present embodiment, a first isolating step is performed to isolate exosomes from a blood sample freed of plasma membrane-derived membrane vesicles and a second isolating step is performed to isolate astrocyte-derived exosomes from other exosomes. In other embodiments, the exosome portion of the exosome-agent complex is lysed using a lysis reagent and the protein levels of the lysed exosome are assayed. In some embodiments, the antibody-exosome complex is created on a solid phase. In yet other embodiments, the methods further comprise releasing the exosome from the antibody-exosome complex. In certain embodiments, the solid phase is non-magnetic beads, magnetic beads, agarose, or sepharose. In other embodiments, the vesicle is released by exposing the antibody-exosome complex to low pH between 3.5 and 1.5. In yet other embodiments, the released exosome is neutralized by adding a high pH solution. In other embodiments, the released exosomes are lysed by incubating the released exosomes with a lysis solution. In still other embodiments, the lysis solution contains inhibitors for proteases and phosphatases.

### Traumatic Brain Injury

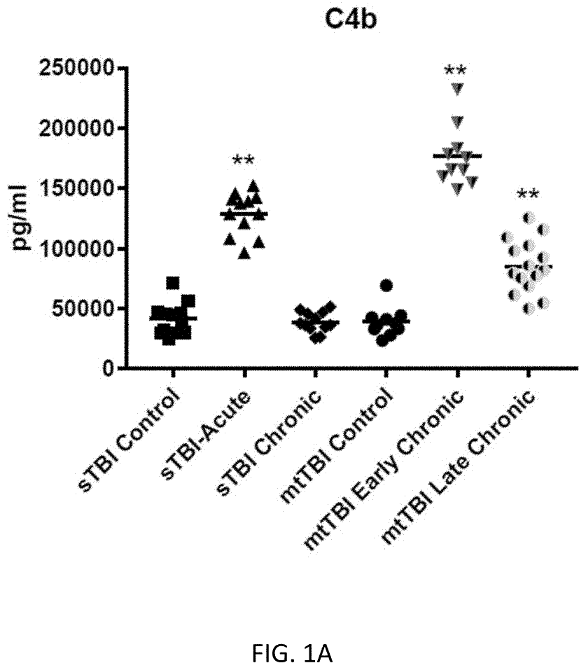

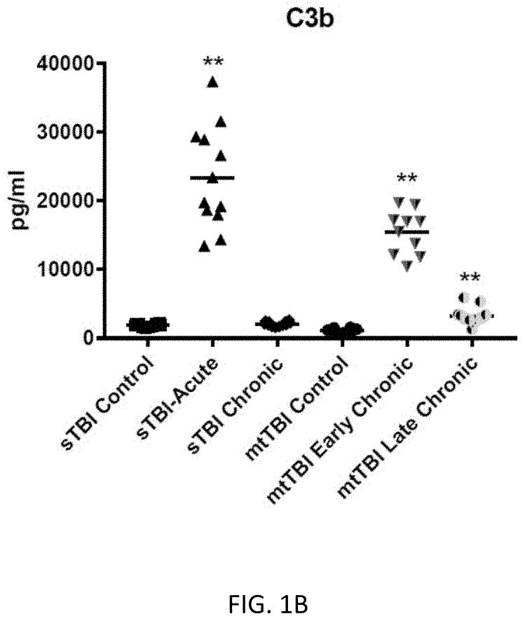

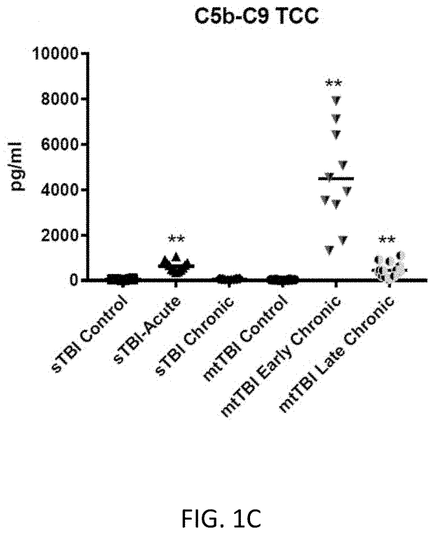

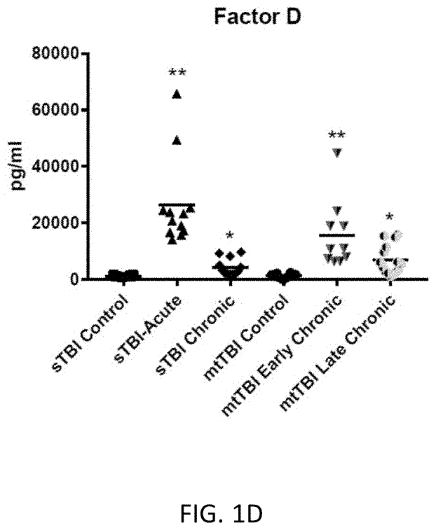

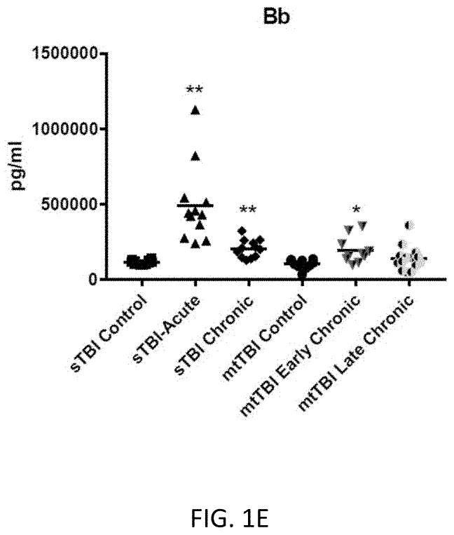

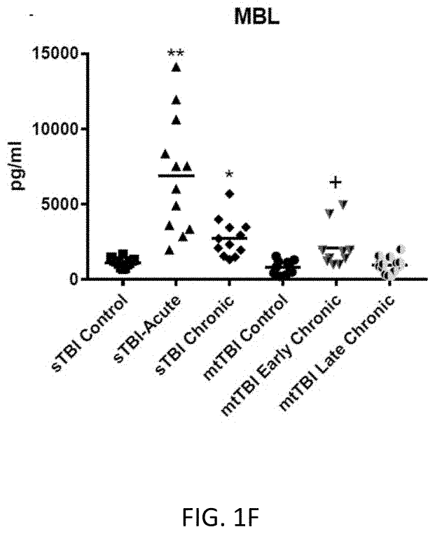

The present invention provides methods for diagnosing a traumatic brain injury in a subject and/or identifying a subject at risk of developing a traumatic brain injury, or prescribing a therapeutic regimen or predicting benefit from therapy. ADE levels of C4b, factor D, Bb and MBL, and the resultant effector components C3b and C5b-9 TCC all are elevated significantly within days and probably hours in acute sTBI relative to controls (see FIG. 1, Example 1). In the chronic phase of sTBI, three months to one year after injury, CD81-normalized ADE levels of alternative pathway factors D and Bb as well as lectin pathway MBL were elevated significantly relative to those of controls (see FIG. 1, Example 1). In early chronic mtTBI, ADE levels of complement proteins in all three pathways and the shared effector proteins C3b and C5b-9 all were increased significantly (see FIG. 1, Example 1). Hence, astrocyte-derived exosomal biomarker abnormalities are associated with development or worsening of a traumatic brain injury. Accordingly, detection of astrocyte-derived exosomal biomarker abnormalities can be used to identify individuals who will benefit from therapy.

In some embodiments, the traumatic brain injury is selected from the group consisting of: acute TBI, chronic TBI, military TBI, and/or sports-related TBI.

In some embodiments, the subject is a mammalian subject, including, e.g., a cat, a dog, a rodent, etc. In certain embodiments, the subject is a human subject.

In some embodiments, the present invention enables a medical practitioner to diagnose or prognose traumatic brain injury in a subject. In yet other embodiments, the present invention enables a medical practitioner to identify a subject at risk of developing a traumatic brain injury. In other embodiments, the present invention enables a medical practitioner to predict whether a subject will later develop a traumatic brain injury. In further embodiments the present invention enables a medical practitioner to prescribe a therapeutic regimen or predict benefit from therapy in a subject having traumatic brain injury or at risk of developing a traumatic brain injury. For example, the administration of one or more recombinant complement control proteins early in traumatic brain injury, guided by their levels in astrocyte-derived exosomes of individual patients, could limit recruitment of complement mechanisms preventatively. In later phases of traumatic brain injury, when complement activation has appeared, neutralizing monoclonal antibodies to effector complement components or their receptors, decoy complement receptors or receptor antagonists, and esterase inhibitors of complement mediator generation may suppress ongoing complement-mediated neuronal injury. Differential diagnosis based on biomarker levels in ADEs isolated from subject with acute TBI, chronic TBI, military TBI, and sports-related TBI are provided by the methods disclosed herein (see Example 1).

### Biomarkers

Astrocyte-derived exosomal cargo levels of biomarker proteins are assayed for a subject having or at-risk of having a traumatic brain injury. In some embodiments, one or more biomarkers selected from the group consisting of an effector complement protein, a membrane-associated complement regulatory protein, an alternative pathway complement protein, a classical pathway complement protein, and a lectin pathway protein are assayed in order to detect whether or not a subject has a traumatic brain injury. In some embodiments, one or more biomarkers selected from the group consisting of human tetraspanning exosome marker CD81, complement fragment C4b, glutamine synthetase, complement receptor type 1 (CR1), factor I, glial acidic fibrillary protein, complement fragment C3b, complement factor B, Bb fragment of complement factor B, terminal complement complex (TCC) C5b-9, CD59, mannose-binding lectin (MBL), and complement factor D are assayed in order to detect whether or not a subject has a traumatic brain injury. In some embodiments, the one or more biomarkers are assayed in the preclinical phase.

One of ordinary skill in the art has several methods and devices available for the detection and analysis of the biomarkers of the instant invention. With regard to polypeptides or proteins in patient test samples, immunoassay devices and methods are often used. These devices and methods can utilize labeled molecules in various sandwich, competitive, or non-competitive assay formats, to generate a signal that is related to the presence or amount of an analyte of interest. Additionally, certain methods and devices, such as biosensors and optical immunoassays, may be employed to determine the presence or amount of analytes without the need for a labeled molecule.

Preferably the markers are analyzed using an immunoassay, although other methods are well known to those skilled in the art (for example, the measurement of marker RNA levels). The presence or amount of a marker is generally determined using antibodies specific for each marker and detecting specific binding. Any suitable immunoassay may be utilized, for example, enzyme-linked immunoassays (ELISA), radioimmunoassay (RIAs), competitive binding assays, planar waveguide technology, and the like. Specific immunological binding of the antibody to the marker can be detected directly or indirectly. Direct labels include fluorescent or luminescent tags, metals, dyes, radionuclides, and the like, attached to the antibody. Indirect labels include various enzymes well known in the art, such as alkaline phosphatase, horseradish peroxidase and the like.

The use of immobilized antibodies specific for the biomarkers is also contemplated by the present invention. The antibodies could be immobilized onto a variety of solid supports, such as magnetic or chromatographic matrix particles, the surface of an assay place (such as microtiter wells), pieces of a solid substrate material (such as plastic, nylon, paper), and the like. An assay strip could be prepared by coating the antibody or a plurality of antibodies in an array on solid support. This strip could then be dipped into the test sample and then processed quickly through washes and detection steps to generate a measurable signal, such as a colored spot.

The analysis of a plurality of biomarkers may be carried out separately or simultaneously with one test sample. Several biomarkers may be combined into one test for efficient processing of a multiple of samples. In addition, one skilled in the art would recognize the value of testing multiple samples (for example, at successive time points) from the same individual. Such testing of serial samples will allow the identification of changes in marker levels over time. Increases or decreases in biomarker levels, as well as the absence of change in biomarker levels, would provide useful information about disease status that includes, but is not limited to the appropriateness of drug therapies, the effectiveness of various therapies, identification of the severity of a traumatic brain injury, susceptibility to traumatic brain injury, and prognosis of the patient's outcome, including risk of development of a traumatic brain injury.

An assay consisting of a combination of the biomarkers referenced in the instant invention may be constructed to provide relevant information related to differential diagnosis. Such a panel may be constructed using 1, 2, 3, 4, 5, 6, 7, 8, 9, 10, 15, 20, or more individual markers. The analysis of a single biomarker or subsets of biomarkers comprising a larger panel of biomarkers could be carried out using methods described within the instant invention to optimize clinical sensitivity or specificity in various clinical settings.

The analysis of markers could be carried out in a variety of physical formats as well. For example, the use of microtiter plates or automation could be used to facilitate the processing of large numbers of test samples. Alternatively, single sample formats could be developed to facilitate immediate treatment and diagnosis in a timely fashion, for example, in ambulatory transport or emergency room settings. Particularly useful physical formats comprise surfaces having a plurality of discrete, addressable locations for the detection of a plurality of different analytes. Such formats include protein microarrays, or “protein chips” and capillary devices.

Biomarkers of the present invention serve an important role in the early detection and monitoring of traumatic brain injury. Biomarkers are typically substances found in a bodily sample that can be measured. The measured amount can correlate with underlying disorder or disease pathophysiology and probability of developing a traumatic brain injury in the future. In patients receiving treatment for their condition, the measured amount will also correlate with responsiveness to therapy.

In some embodiments, the biomarker is measured by a method selected from the group consisting of immunohistochemistry, immunocytochemistry, immunofluorescence, immunoprecipitation, western blotting, and ELISA.

### Clinical Assay Performance

The methods of the present invention for detecting traumatic brain injury may be used in clinical assays to diagnose or prognose a traumatic brain injury in a subject, identify a subject at risk of a traumatic brain injury, and/or for prescribing a therapeutic regimen or predicting benefit from therapy in a subject having a traumatic brain injury. Clinical assay performance can be assessed by determining the assay's sensitivity, specificity, area under the ROC curve (AUC), accuracy, positive predictive value (PPV), and negative predictive value (NPV). Disclosed herein are assays for diagnosing or prognosing a traumatic brain injury in a subject, identifying a subject at risk of a traumatic brain injury, or for prescribing a therapeutic regimen or predicting benefit from therapy in a subject having a traumatic brain injury.

The clinical performance of the assay may be based on sensitivity. The sensitivity of an assay of the present invention may be at least about 40%, 45%, 50%, 55%, 60%, 65%, 70%, 75%, 80%, 85%, 90%, 95%, 99%, or 100%. The clinical performance of the assay may be based on specificity. The specificity of an assay of the present invention may be at least about 40%, 45%, 50%, 55%, 60%, 65%, 70%, 75%, 80%, 85%, 90%, 95%, 99%, or 100%. The clinical performance of the assay may be based on area under the ROC curve (AUC). The AUC of an assay of the present invention may be at least about 0.5, 0.55, 0.6, 0.65, 0.7, 0.75, 0.8, 0.85, 0.9, or 0.95. The clinical performance of the assay may be based on accuracy. The accuracy of an assay of the present invention may be at least about 40%, 45%, 50%, 55%, 60%, 65%, 70%, 75%, 80%, 85%, 90%, 95%, 99%, or 100%.

### Agents and Compositions

Agents and compositions useful in the methods of the present invention include agents and compositions that specifically recognize one or more astrocyte-derived exosomal biomarkers associated with traumatic brain injury, including an effector complement protein, a membrane-associated complement regulatory protein, an alternative pathway complement protein, a classical pathway complement protein, and/or a lectin pathway protein or any combination thereof. In some embodiments, the one or more astrocyte-derived exosomal biomarker is human tetraspanning exosome marker CD81, complement fragment C4b, glutamine synthetase, complement receptor type 1 (CR1), factor I, glial acidic fibrillary protein, complement fragment C3b, complement factor B. Bb fragment of complement factor B, terminal complement complex (TCC) C5b-9, CD59, mannose-binding lectin (MBL), and/or complement factor D or any combination thereof. In some embodiments, the agent or composition enhances the activity of at least one biomarker. In other embodiments, the agent or composition decreases the activity of at least one biomarker. In some embodiments, the agent composition increases the levels of at least one biomarker in the subject. In other embodiments, the agent composition decreases the levels of at least one biomarker in the subject. In yet other embodiments, the agent composition comprises a peptide, a nucleic acid, an antibody, or a small molecule.

In certain embodiments, the present invention relates to agents and/or compositions that specifically detect a biomarker associated with traumatic brain injury. As detailed elsewhere herein, the present invention is based upon the finding that astrocyte-derived exosomal complement proteins including, for example, effector proteins (e.g., C3b), membrane-associated complement regulatory proteins (CD59), alternative pathway proteins (e.g., factor D), classical pathway proteins (e.g., C4b), and lectin pathway proteins (e.g., MBL) are specific biomarkers for traumatic brain injury. In one embodiment, the compositions of the invention specifically bind to and detect one or more of the following biomarkers: human tetraspanning exosome marker CD81, complement fragment C4b, glutamine synthetase, complement receptor type 1 (CR1), factor 1, glial acidic fibrillary protein, complement fragment C3b, complement factor B, Bb fragment of complement factor B, terminal complement complex (TCC) C5b-9, CD59, mannose-binding lectin (MBL), and complement factor D, or any combination thereof. The agent and/or composition of the present invention can comprise an antibody, a peptide, a small molecule, a nucleic acid, and the like.

In some embodiments, the agent and/or composition comprises an antibody, wherein the antibody specifically binds to a biomarker or astrocyte-derived exosomes. The term “antibody” as used herein and further discussed below is intended to include fragments thereof which are also specifically reactive with a biomarker or vesicle (e.g., exosome). Antibodies can be fragmented using conventional techniques and the fragments screened for utility in the same manner as described above for whole antibodies. For example, F(ab)₂ fragments can be generated by treating antibody with pepsin. The resulting F(ab)₂ fragment can be treated to reduce disulfide bridges to produce Fab fragments. Antigen-binding portions may also be produced by recombinant DNA techniques or by enzymatic or chemical cleavage of intact antibodies. Antigen-binding portions include, inter alia, Fab, Fab′, F(ab′)₂, Fv, dAb, and complementarity determining region (CDR) fragments, single-chain antibodies (scFv), single domain antibodies, bispecific antibodies, chimeric antibodies, humanized antibodies, diabodies and polypeptides that contain at least a portion of an immunoglobulin that is sufficient to confer specific antigen binding to the polypeptide. In certain embodiments, the antibody further comprises a label attached thereto and able to be detected (e.g., the label can be a radioisotope, fluorescent compound, enzyme or enzyme co-factor).

In certain embodiments, an antibody of the invention is a monoclonal antibody, and in certain embodiments, the invention makes available methods for generating novel antibodies that specifically bind the biomarker or the exosome of the invention. For example, a method for generating a monoclonal antibody that specifically binds a biomarker or exosome, may comprise administering to a mouse an amount of an immunogenic composition comprising the biomarker or exosome, or fragment thereof, effective to stimulate a detectable immune response, obtaining antibody-producing cells (e.g., cells from the spleen) from the mouse and fusing the antibody-producing cells with myeloma cells to obtain antibody-producing hybridomas, and testing the antibody-producing hybridomas to identify a hybridoma that produces a monocolonal antibody that binds specifically to the biomarker or exosome. Once obtained, a hybridoma can be propagated in a cell culture, optionally in culture conditions where the hybridoma-derived cells produce the monoclonal antibody that binds specifically to the biomarker or exosome. The monoclonal antibody may be purified from the cell culture.

The term “specifically reactive with” or “specifically binds” as used in reference to an antibody is intended to mean, as is generally understood in the art, that the antibody is sufficiently selective between the antigen of interest (e.g., a biomarker or exosome) and other antigens that are not of interest. In certain methods employing the antibody, such as therapeutic applications, a higher degree of specificity in binding may be desirable. Monoclonal antibodies generally have a greater tendency (as compared to polyclonal antibodies) to discriminate effectively between the desired antigens and cross-reacting polypeptides. One characteristic that influences the specificity of an antibody:antigen interaction is the affinity of the antibody for the antigen. Although the desired specificity may be reached with a range of different affinities, generally preferred antibodies will have an affinity (a dissociation constant) of about 10⁻⁶, 10⁻⁷, 10⁻⁸, 10⁻⁹ or less.

Antibodies can be generated to bind specifically to an epitope of an astrocyte-derived exosome or a biomarker of the present invention, including, for example, astrocyte-derived exosome surface markers, such as Glutamine Aspartate Transporter (GLAST).

In addition, the techniques used to screen antibodies in order to identify a desirable antibody may influence the properties of the antibody obtained. A variety of different techniques are available for testing interaction between antibodies and antigens to identify particularly desirable antibodies. Such techniques include ELISAs, surface plasmon resonance binding assays (e.g., the Biacore binding assay, Biacore AB, Uppsala, Sweden), sandwich assays (e.g., the paramagnetic bead system of IGEN International, Inc., Gaithersburg, Md.), western blots, immunoprecipitation assays, immunocytochemistry, and immunohistochemistry.

In some embodiments, the present invention relates to agent and/or compositions used for treating or preventing a traumatic brain injury. As detailed elsewhere herein, abnormal levels of astrocyte-derived exosomal biomarkers are implicated in the pathology of traumatic brain injury. Therefore, in one embodiment, the present invention provides compositions that inhibit or reduce abnormalities in levels of astrocyte-derived exosomal biomarkers. Compositions and agents useful for preventing and/or reducing abnormalities in levels of astrocyte-derived exosomal biomarkers may include proteins, peptides, nucleic acids, small molecules, and the like. In some embodiments, the agent is a complement pathway inhibitor. In other embodiments, the complement pathway inhibitor is a neutralizing monoclonal antibody to effector complement components or their receptors, decoy complement receptors or receptor antagonists, or an esterase inhibitor.

### Methods of Treatment

The present invention provides methods for treating traumatic brain injury in a subject, the method comprising administering an effective amount of a complement pathway inhibitor to a subject having or suspected of having a traumatic brain injury, thereby treating the traumatic brain injury.

Generally, the therapeutic/diagnostic agents used in the invention are administered to a subject in an effective amount. Generally, an effective amount is an amount effective to (1) reduce the symptoms of the traumatic brain injury to be treated, (2) induce a pharmacological change relevant to treating the traumatic brain injury to be treated or (3) detect traumatic brain injury in vivo or in vitro. For example, an effective amount of an agent of the invention includes an amount effective to: prevent or reduce cognitive impairment in a subject with or suspected of having a traumatic brain injury.

Effective amounts of the agents can be any amount or dose sufficient to bring about the desired effect and will depend, in part, on the condition, type and location of the traumatic brain injury, the size and condition of the patient, as well as other factors readily known to those skilled in the art. The dosages can be given as a single dose, or as several doses, for example, divided over the course of several weeks. It is specifically contemplated that complement inhibitors may need to be administered immediately (or as soon as possible) and for several months following a brain injury to be optimally effective.

The invention is also directed toward methods of treatment utilizing the therapeutic compositions of the present invention. The method comprises administering the therapeutic agent to a subject in need of such administration, such as, for example, a subject with a traumatic brain injury.

The therapeutic agents of the instant invention can be administered by any suitable means as described herein, including, for example, parenteral, topical, oral or local administration, such as intradermally, by injection, or by aerosol. In one embodiment of the invention, the agent is administered by injection. Such injection can be locally administered to any affected area. A therapeutic composition can be administered in a variety of unit dosage forms depending upon the method of administration. For example, unit dosage forms suitable for oral administration of a subject include powder, tablets, pills and capsules. Preferred delivery methods for a therapeutic composition of the invention include intravenous administration and local administration by, for example, injection or topical administration. For particular modes of delivery, a therapeutic composition of the invention can be formulated in an excipient of the invention. A therapeutic agent of the invention can be administered to any subject, to mammals, and to humans.

The particular mode of administration will depend on the traumatic brain injury to be treated. It is contemplated that administration of the agents of the present invention may be via any bodily fluid, or any target or any tissue accessible through a body fluid.

In some embodiments, the traumatic brain injury (TBI) is acute TBI, chronic TBI, military TBI, and/or sports-related TBI. In other embodiments, the complement pathway inhibitor is a neutralizing monoclonal antibody to effector complement components or their receptors, decoy complement receptors or receptor antagonists, or an esterase inhibitor. Complement inhibitors that may be used in the methods of the invention include the complement inhibitors disclosed in United States Patent Application Publication Number 2016/0096870, the contents of which are incorporated by reference in its entirety.

Furthermore, the methods of the invention can be used for monitoring the efficacy of therapy in a patient. The method comprises: analyzing the levels of one or more biomarkers selected from the group consisting of inflammatory cytokines, interleukin 6 (IL-6), tumor necrosis factor alpha (TNF-α), interleukin 1 beta (IL-10), complement proteins, C1q, C4b, C5b, C3d, factor B, factor D, Fragment Bb, C3b, C5b-C9 terminal complement complex (TCC), complement regulatory proteins, membrane inhibitor of reactive lysis (CD59), membrane cofactor protein (CD46), decay-accelerating factor (DAF) and complement receptor type 1 (CR1) from astrocyte-derived exosomes from biological samples from the patient before and after the patient undergoes the therapy, in conjunction with respective reference levels for the biomarkers. Increasing levels of inflammatory cytokines, interleukin 6 (IL-6), tumor necrosis factor alpha (TNF-α), interleukin 1 beta (IL-1p), complement proteins, C1q, C4b, C5b, C3d, factor B, factor D, Fragment Bb, C3b, and C5b-C9 terminal complement complex (TCC) astrocyte-derived exosomal biomarkers correlate with increased traumatic brain injury severity and indicate that the patient is worsening or not responding to the therapy, and decreasing levels of inflammatory cytokines, interleukin 6 (IL-6), tumor necrosis factor alpha (TNF-α), interleukin 1 beta (IL-1β), complement proteins, C1q, C4b, C5b, C3d, factor B, factor D, Fragment Bb, C3b, and C5b-C9 terminal complement complex (TCC) astrocyte-derived exosomal biomarkers correlate with reduced traumatic brain injury severity and indicate that the condition of the patient is improving. Decreasing levels of complement regulatory proteins, membrane inhibitor of reactive lysis (CD59), membrane cofactor protein (CD46), decay-accelerating factor (DAF) and complement receptor type 1 (CR1) astrocyte-derived exosomal biomarkers correlate with increased traumatic brain injury severity and indicate that the patient is worsening or not responding to the therapy, and increasing levels of complement regulatory proteins, membrane inhibitor of reactive lysis (CD59), membrane cofactor protein (CD46), decay-accelerating factor (DAF) and complement receptor type 1 (CR1) astrocyte-derived exosomal biomarkers correlate with reduced traumatic brain injury severity and indicate that the condition of the patient is improving.

In some embodiments, the methods of the invention provide a method for treating traumatic brain injury the method comprising the steps of: obtaining a biological sample from a subject suspected of having a traumatic brain injury, wherein the sample comprises astrocyte-derived exosomes; measuring the level of one or more biomarkers selected from the group consisting of inflammatory cytokines, interleukin 6 (IL-6), tumor necrosis factor alpha (TNF-α), interleukin 1 beta (IL-10), complement proteins, C1q, C4b, C5b, C3d, factor B, factor D, Fragment Bb, C3b, C5b-C9 terminal complement complex (TCC), complement regulatory proteins, membrane inhibitor of reactive lysis (CD59), membrane cofactor protein (CD46), decay-accelerating factor (DAF) and complement receptor type 1 (CR1) from the biological sample, wherein an altered level of the one or more biomarkers in the sample relative to the level in a control sample is indicative of a need for treatment; and administering an effective amount of an agent to the subject thereby treating the traumatic brain injury in the subject.

### Kits

Another aspect of the invention encompasses kits for detecting or monitoring traumatic brain injury in a subject. A variety of kits having different components are contemplated by the current invention. Generally speaking, the kit will include the means for quantifying one or more biomarkers in a subject. In another embodiment, the kit will include means for collecting a biological sample, means for quantifying one or more biomarkers in the biological sample, and instructions for use of the kit contents. In certain embodiments, the kit comprises a means for enriching or isolating astrocyte-derived exosomes in a biological sample. In further aspects, the means for enriching or isolating astrocyte-derived exosomes comprises reagents necessary to enrich or isolate astrocyte-derived exosomes from a biological sample. In certain aspects, the kit comprises a means for quantifying the amount of a biomarker. In further aspects, the means for quantifying the amount of a biomarker comprises reagents necessary to detect the amount of a biomarker.

These and other embodiments of the present invention will readily occur to those of ordinary skill in the art in view of the disclosure herein.

### EXAMPLES

The invention will be further understood by reference to the following examples, which are intended to be purely exemplary of the invention. These examples are provided solely to illustrate the claimed invention. The present invention is not limited in scope by the exemplified embodiments, which are intended as illustrations of single aspects of the invention only. Any methods that are functionally equivalent are within the scope of the invention. Various modifications of the invention in addition to those described herein will become apparent to those skilled in the art from the foregoing description. Such modifications are intended to fall within the scope of the appended claims.

### Example 1: Traumatic Brain Injury Alters Plasma Astrocyte-Derived Exosome Levels of Complement Proteins

Neurotoxic complement proteins were detected and measured in astrocyte-derived exosomes from subjects with traumatic brain injury as follows. Blood samples were obtained from subjects with sports-related (sTBI, including chronic sTBI) or military TBI (mtTBI, both early chronic and late chronic mtTBI) and gender-matched control subjects. Chronic sTBI patients were defined as having had at least two TBIs, the most recent of which was no less than three months and no more than 12 months before blood collection. Blood was collected from early chronic mtTBI subjects one to four years after their TBI, whereas late chronic mtTBI subjects (n=15) provided blood 12 years or more after their TBI (Table 1). mtTBI was defined as a head injury to a military subject resulting in any form of medical care.

Ten milliliters of venous blood were drawn into 0.5 ml saline with EDTA, incubated for 10 minutes at room temperature, and centrifuged for 15 minutes at 2500×g. Plasmas were stored in 0.25-ml aliquots at −80° C.

Aliquots of 0.25 ml plasma were incubated with 0.1 ml thromboplastin D (Thermo Fisher Scientific, Waltham, Mass., USA), followed by addition of 0.15 ml of calcium- and magnesium-free Dulbecco's balanced salt solution with protease inhibitor cocktail (Roche, Indianapolis, Ind., USA) and phosphatase inhibitor cocktail (Thermo Fisher Scientific) (DBS⁺⁺). After centrifugation at 3000×g for 30 min at 4° C., total exosomes were harvested from resultant supernatants by 126 uL per tube of ExoQuick (System Biosciences, Mountain View, Calif., USA) and centrifugation at 1.500×g for 30 minutes at 4° C. To enrich ADEs, total exosomes were resuspended in 0.35 ml of DBS- and incubated for 60 minutes at room temperature with 2.0 ug of mouse anti-human glutamine aspartate transporter (ACSA-1) biotinylated antibody (Miltenyi Biotec, Auburn, Calif.) in 50 ul of 3% bovine serum albumin (BSA; 1:3.33 dilution of Blocker BSA 10% solution in DBS⁻: Thermo Fisher Scientific) per tube with mixing, followed by addition of 10 ul of streptavidin-agarose Ultralink resin (Thermo Fisher Scientific) in 40 ul of 3% BSA and incubation for 30 minutes at room temperature with mixing (Goetzl et al. (2016) Faseb J 30, 3853-3859). After centrifugation at 800×g for 10 minutes at 4° C. and removal of the supernatant, each pellet was suspended in 100 ul of cold 0.05M glycine-HCl (pH 3.0) by gentle mixing for 10 seconds and centrifuged at 4,000×g for 10 minutes, all at 4° C. Supernatants then were transferred to clean tubes containing 25 ul of 10% BSA and 10 ul of 1M Tris-HCl (pH 8.0) and mixed before addition of 365 ul of mammalian protein extraction reagent (M-PER, Thermo Fisher Scientific). Resultant 0.5 ml lysates of ADEs were stored at −80° C. NDEs were prepared as described in Goetzl et al. (2016) Faseb J 30, 4141-4148.

ADE and NDE proteins were quantified by enzyme-linked immunosorbent assay (ELISA) kits for human tetraspanning exosome marker CD81, complement fragment C4b (ARP American Research Products, Waltham, Mass.; Cusabio Technology, College Park, Md.), glutamine synthetase, complement receptor type 1 (CR1), factor I (ARP American Research Products; Cloud-Clone Corp, Katy, Tex.), glial acidic fibrillary protein (EMD Millipore, Billerica, Mass.), complement fragment C3b, complement factor B (Abcam, Cambridge, Mass.). Bb fragment of complement factor B (Quidel-Microvue, San Diego, Calif.), terminal complement complex (TCC) C5b-9 (Aviva Systems. San Diego, Calif.), CD59, mannose-binding lectin (MBL) (Ray Biotech, Norcross, Ga.), and complement factor D (Thermo Fisher Scientific-Invitrogen, LaFayette, Colo.). The mean value for all determinations of CD81 in each assay group was set at 1.00, and relative values of CD81 for each sample were used to normalize their recovery.

A Shapiro-Wilks test showed that data in all 54 sets, except three, were distributed normally. Statistical significance of differences between means for TBI groups and normal controls were determined with an unpaired Student t test, including a Bonferroni correction. For the three non-normally distributed sets, significance was determined by a Mann-Whitney U test.

The ages and sex distribution were the same for participants of the control, acute and chronic sTBI groups (Table 1). Subsequently determined ADE data were indistinguishable for the early and late chronic mtTBI control groups, so results for these 10 subjects were considered together as one control group for both the early and late mtTBI subjects. Sex distribution was the same for the control and early chronic mtTBI groups, but men were over-represented in the late chronic mtTBI group (Table 1). No subject in any group had cognitive impairment.

TABLE 1
Demographics and TBI History of Participants

| | | | | Interval between |
|---|---|---|---|---|
| | | | | last TBI and blood |
| | Age | Sex | Total | donation |
| A. sTBI* | (mean ± SEM) | (female/male) | TBIs-Subjects | (mean ± SEM) |
| Control (n = 12) | 21.6 ± 0.68 | 6/6 | None | — |
| Acute TBI (n = 12) | 21.3 ± 0.40 | 6/6 | 1-5 | ≤1 week |
| | | | 2-1 | |
| Chronic TBI (n = 12) | 20.1 ± 0.48 | 6/6 | 2-6 | 6.58 ± 0.89 |
| | | | 3-3 | months |
| | | | 4-3 | |
| | | | | Months between |
| | | | | last TBI and blood |
| | | | | donation |
| B. mtTBI⁺ | | | | (mean ± SEM) |
| Chronic control | | | | |
| Early (n = 5) | 38.0 ± 3.89 | 1/4 | None | — |
| Late (n = 5) | 74.6 ± 1.81 | 2/3 | None | — |
| Chronic TBI | | | | |
| Early (n = 10) | 38.4 ± 2.44 | 3/7 | 1-7 | 28.2 ± 4.74 |
| | | | 2-3 | |
| Late (n = 15) | 77.0 ± 1.86 | 2/13 | 1-4 | 561 ± 58.4 |
| | | | 2-5 | |
| | | | 3-3 | |
| | | | 4-3 | |

sTBI, sports-related TBI;
mtTBI, military-inflicted TBI;
*acute sTBI participants had no previous episodes of TBI or up to 2 previous episodes at least 12 months before the study episode, whereas chronic sTBI participants had at least 2 previous episodes with the most recent being no more than 12 months before the study episode;
⁺early chronic mtTBI encompasses participants studied 1-4 years after TBI and late chronic mtTBI those studied ≥12 years after TBI.

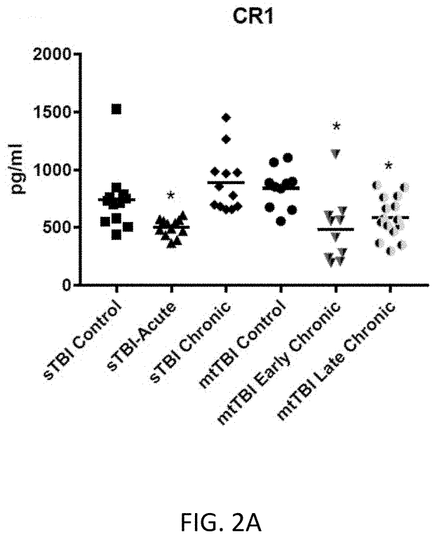

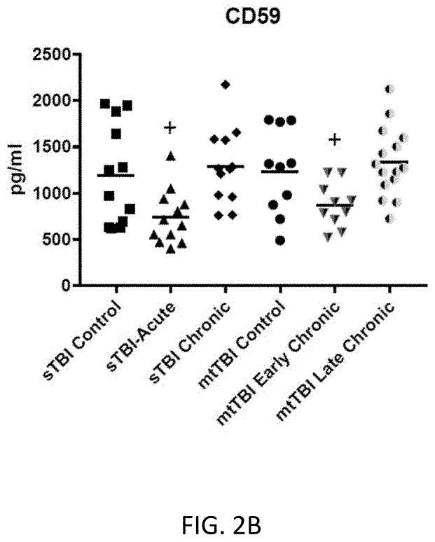

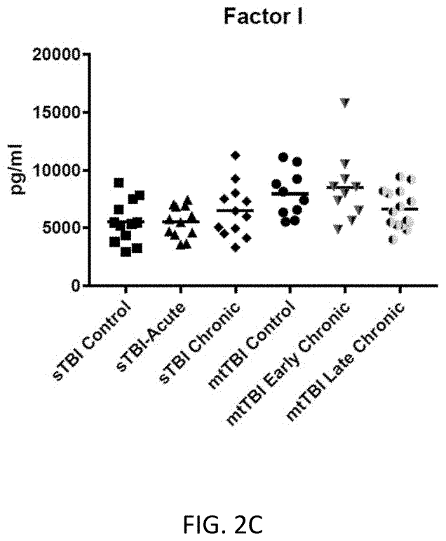

Extracellular vesicle complement proteins levels for the subjects in an acute phase of sTBI were significantly higher in ADEs than NDEs (Table 2). Plasma levels of ADEs determined by counts and CD81 content were significantly lower for the acute sTBI group, but not any of the chronic TBI groups, relative to those of corresponding controls (Table 3). Therefore, all values for ADE complement proteins in FIGS. 1 and 2 were normalized for CD81 content.

TABLE 2
Differences in Levels of Complement Proteins
in ADEs and NDEs of Acute sTBI

| | C4b | Factor D | Bb | C3b | C5b-9 TCC |
|---|---|---|---|---|---|
| ADEs | 124,208 ± | 25,341 ± | 606,352 ± | 23,798 ± | 687 ± |
| | 7469 | 5116 | 128,438 | 3916 | 96.1 |
| NDEs | 9769 ± | 960 ± | 17,130 ± | 1310 ± | 19.4 ± |
| | 732 | 68.6 | 2120 | 53.6 | 2.05 |

All values are mean pg/ml ± S.E.M. for plasmas of six sTBI subjects in the acute phase after injury.
Differences all are significant at p < 0.001.

TABLE 3
Acute Decrease in Plasma ADE Concentration After TBI

| A. sTBI | Control | Acute | Chronic |
|---|---|---|---|
| CD81 | 1250 ± 36.7 | 959 ± 86.7* | 1311 ± 61.3 |
| (pg/ml) | | | |
| Counts | 56.0 ± 1.75 | 43.8 ± 1.78** | 56.5 ± 1.41 |
| (×10⁹/ml) | | | |
| B. mtTBI | Control | Early Chronic | Late Chronic |
| CD81 | 1658 ± 264 | 1160 ± 38.6 | 1282 ± 42.2 |
| (pg/ml) | | | |
| Counts | 60.7 ± 1.33 | 56.5 ± 1.33 | 58.7 ± 1.33 |
| (×10⁹/ml) | | | |

sTBI, sports-related TBI;
mtTBI, military-inflicted TBI;
unpaired t test comparison to control value with *p < 0.01 and **p < 0.001.

In the acute phase of sTBI, for example, within one week of injury, CD81-normalized ADE levels of complement proteins C4b, factor D, Bb and MBL of all three pathways as well as shared effector proteins C3b and C5b-9 TCC were increased significantly relative to those of controls (see FIG. 1) and those of the membrane-associated complement regulatory proteins CR1 and CD59 (see FIG. 2), but not fluid-phase factor I, were decreased significantly relative to those of controls. In the chronic phase of sTBI, three months to one year after injury, CD81-normalized ADE levels of alternative pathway factors D and Bb as well as lectin pathway MBL, but not of classical pathway C4b or the effector proteins C3b and C5b-9 TCC, were elevated significantly relative to those of controls (see FIG. 1). None of the CD81-normalized ADE levels of any of the complement regulatory proteins were decreased in chronic sTBI (see FIG. 2). In early chronic mtTBI, one to four years after injury, ADE levels of complement proteins in all three pathways and the shared effector proteins C3b and C5b-9 all were increased significantly (FIG. 1) and of the membrane-associated complement regulatory proteins CR1 and CD59, but not factor 1, were decreased significantly (FIG. 2) relative to those of controls. In late chronic mtTBI, 12 or more years after injury, ADE levels of complement proteins C4b, factor D, and the shared effector proteins C3b and C5b-9, but not of Bb or MBL, were increased significantly (see FIG. 1) and of the membrane-associated complement regulatory protein CR1, but not CD59 or factor I, was decreased significantly (see FIG. 2) relative to those of controls. Therefore, generation of the neurotoxic complement factors C3b and C5b-9 by one or more pathways in TBI begins as soon as days after injury and may continue for years after TBI depending on the nature and severity of the injury.

The maximal increases in C3b and C5b-9 TCC observed in acute sTBI were no longer sustained months later in chronic sTBI, along with subsidence of the acute increases in C4b, factor D, Bb and MBL (see FIG. 1). For early chronic mtTBI with post-TBI intervals similar to chronic sTBI, the ADE levels of C3b, C5b-9 TCC, C4b, factor D, Bb and MBL all were at their highest point for the mtTBI set and C3b, C5b-9 TCC, C4b and factor D remained elevated in late chronic mtTB1. All episodes of sTBI were mild, whereas some episodes of mtTBI were moderate. However, there was no correlation between the clinical severity of mtTBI and the level of complement effector proteins in early chronic mtTBI.

In acute sTBI, all three complement pathways appear to contribute rapidly to the increased ADE levels of C3b and C5b-9 TCC that are capable of damaging synapses and injuring neurons. The ADE levels of C4b, factor D. Bb and MBL, and the resultant effector components C3b and C5b-9 TCC all are elevated significantly within days and probably hours in acute sTBI relative to controls (FIG. 1). Similarly, ADE levels of the complement regulatory membrane proteins CR1 and CD59 decline within days after TBI (FIG. 2). In contrast, the time-courses of correction of abnormal ADE levels of complement proteins in TBI is extremely variable, presumably in relation to the type, severity and presumably other characteristics of the injury. Return of ADE levels of C4b. C3b, C5b-9 TCC, CR1 and CD59, but not factor D, Bb or MBL, to normal was complete in sTBI within months. In contrast, return of ADE levels of Bb, MBL and CD59 to those of controls in mtTBI required many years and normalization of ADE levels of C4b, C3b, C5b-9 TCC and CR1 was not complete decades after injury.

These results showed that ADE levels of complement proteins are altered in subjects with traumatic brain injury. These results demonstrated that the methods of the present invention are useful for detecting biomarkers and measuring biomarker protein levels in astrocyte-derived exosomes. These results further demonstrated that the methods of the present invention may be used to detect exosomal complement biomarkers associated with pathogenesis of neurological diseases, including traumatic brain injury. These results further showed that methods of the present invention are useful for prognosis, diagnosis, treating or monitoring treatment of exosomal complement abnormalities associated with traumatic brain injury. The results suggested that the methods of the present invention would be useful for treating traumatic brain injury.

Various modifications of the invention, in addition to those shown and described herein, will become apparent to those skilled in the art from the foregoing description. Such modifications are intended to fall within the scope of the appended claims.

All references cited herein are hereby incorporated by reference herein in their entirety.

What is claimed is:

1. A method comprising: a) providing a biological sample comprising astrocyte-derived exosomes from a subject having or suspected of having a traumatic brain injury; b) enriching the sample for astrocyte-derived exosomes; and c) detecting the presence of one or more biomarkers selected from the group consisting of an effector complement protein, a membrane-associated complement regulatory protein, an alternative pathway complement protein, a classical pathway complement protein, and/or a lectin pathway protein in the sample, thereby detecting the presence of one or more biomarkers in a biological sample from a subject having or suspected of having a traumatic brain injury.

2. The method of claim 1, wherein the one or more marker is human tetraspanning exosome marker CD81, complement fragment C4b, glutamine synthetase, complement receptor type 1 (CR1), factor I, glial acidic fibrillary protein, complement fragment C3b, complement factor B, Bb fragment of complement factor B, terminal complement complex (TCC) C5b-9, CD59, mannose-binding lectin (MBL), and/or complement factor D.

3. The methods of claim 1, wherein the biological sample is selected from the list consisting of whole blood, plasma, serum, lymph, amniotic fluid, urine, and saliva.

4. The method of claim 1, wherein the detecting the presence of the marker in the biological sample comprises detecting the amount of the marker in the biological sample.

5. The method of claim 1, further comprising the step of determining a treatment course of action based on the detection of the one or more biomarkers.

6. The method of claim 1, wherein the traumatic brain injury (TBI) is acute TBI, chronic TBI, military TBI, and/or sports-related TBI.

7. A method comprising: a) providing a biological sample comprising astrocyte-derived exosomes from a subject having a traumatic brain injury or suspected of having a traumatic brain injury; b) isolating astrocyte-derived exosomes from the biological sample; and c) detecting the presence of one or more biomarkers selected from the group consisting of an effector complement protein, a membrane-associated complement regulatory protein, an alternative pathway complement protein, a classical pathway complement protein, and/or a lectin pathway protein in the exosomes.

8. The method of claim 7, wherein the isolating astrocyte-derived exosomes from the biological sample comprises: contacting the biological sample with an agent under conditions wherein an astrocyte-derived exosome present in the biological sample binds to the agent to form an astrocyte-derived exosome-agent complex; and isolating the astrocyte-derived exosome from the astrocyte-derived exosome-agent complex to obtain a sample containing the astrocyte-derived exosome, wherein the purity of the astrocyte-derived exosomes present in said sample is greater than the purity of the astrocyte-derived exosomes present in said biological sample.

9. The method of claim 7, wherein the one or more marker is human tetraspanning exosome marker CD81, complement fragment C4b, glutamine synthetase, complement receptor type 1 (CR1), factor 1, glial acidic fibrillary protein, complement fragment C3b, complement factor B, Bb fragment of complement factor B, terminal complement complex (TCC) C5b-9, CD59, mannose-binding lectin (MBL), and/or complement factor D.

10. The method of claim 8, wherein the agent is an antibody.

11. The method of claim 10, wherein the antibody is an anti-Glutamine Aspartate Transporter antibody.

12. The methods of claim 7, wherein the biological sample is selected from the list consisting of whole blood, plasma, serum, lymph, amniotic fluid, urine, and saliva.

13. The method of claim 7, wherein the detecting the presence of the marker in the biological sample comprises detecting the amount of the marker in the biological sample.

14. The method of claim 7, further comprising the step of determining a treatment course of action based on the detection of the one or more biomarkers.

15. The method of claim 7, wherein the traumatic brain injury (TBI) is acute TBI, chronic TBI, military TBI, and/or sports-related TBI.

16. A method for treating a subject, comprising the steps of: providing a biological sample from a subject having or suspected of having a traumatic brain injury, wherein the sample comprises astrocyte-derived exosomes; measuring the level of one or more biomarkers selected from the group consisting of an effector complement protein, a membrane-associated complement regulatory protein, an alternative pathway complement protein, a classical pathway complement protein, and/or a lectin pathway protein from the biological sample, wherein an altered level of the one or more biomarkers in the sample relative to the level in a control sample is indicative of a need for treatment; and administering an effective amount of an agent to the subject thereby treating the traumatic brain injury in the subject.

17. The method of claim 16, wherein the one or more marker is human tetraspanning exosome marker CD81, complement fragment C4b, glutamine synthetase, complement receptor type 1 (CR1), factor I, glial acidic fibrillary protein, complement fragment C3b, complement factor B, Bb fragment of complement factor B, terminal complement complex (TCC) CSb-9, CD59, mannose-binding lectin (MBL), and/or complement factor D.

18. The method of claim 16, wherein the traumatic brain injury (TBI) is acute TBI, chronic TBI, military TBI, and/or sports-related TBI.

19. The method of claim 16, wherein the agent is a complement pathway inhibitor.

20. The method of claim 19, wherein the complement pathway inhibitor is a neutralizing monoclonal antibody to effector complement components or their receptors, decoy complement receptors or receptor antagonists, or an esterase inhibitor.
